(19) United States
(12) Patent Application Publication
(10) Pub. No.: US 2024/0099891 A1
(43) Pub. Date: Mar. 28, 2024

(54) DEVICES FOR PREVENTING EAR PRESSURE INJURIES AND METHODS OF MAKING AND USING SAME

(71) Applicant: Cecilia Yanez, Darien, IL (US)

(72) Inventor: Cecilia Yanez, Darien, IL (US)

(21) Appl. No.: 17/953,254

(22) Filed: Sep. 26, 2022

(51) Int. Cl. A61F 11/14; A42B 7/00

(52) U.S. Cl. CPC A61F 11/14; A42B 7/00

(57) ABSTRACT

The present disclosure provides devices for preventing ear lobe pressure injuries in a subject, and methods of making and using same.

### PRIORITY CLAIM

This application claims priority to U.S. Provisional Patent Application Ser. No. 63/147,213, filed on Feb. 8, 2021, the entire contents of which are incorporated herein by reference and relied upon.

### BACKGROUND

Pressure ulcers or pressure injuries are generally defined as localized injuries to the skin and/or underlying tissue usually over a bony prominence, as a result of pressure, or pressure in combination with shear and/or friction. Such injuries are common in hospital or hospice settings, especially among patients who lack the strength, coordination, or consciousness to reposition their bodies at a frequency that avoids development of these types of wounds.

Current strategies for avoiding pressure ulcers or pressure injuries predominantly entail moving the patient periodically, maintaining sufficient hydration levels, and cleaning the patient's skin thoroughly and regularly. However, these strategies frequently fail to prevent pressure injuries on highly sensitive areas where skin is relatively thin, and tissue between the skin and underlying bone or cartilage is relatively minimal. For example, skin of the ear is particularly susceptible to pressure wound development because little tissue separates the skin from ear cartilage.

In view of these clinically relevant shortcomings in existing technologies, a need for improved devices for avoiding pressure ulcers or pressure injuries persists.

### SUMMARY

The present disclosure provides devices for preventing ear lobe pressure injuries in a subject, and methods of making and using same.

In some embodiments, the present disclosure provides a device for preventing a pressure injury associated with an ear of a subject, the device comprising: a first cushion configured to substantially surround a first ear of the subject; a first retention band associated with the first cushion and configured to secure the first cushion to a jaw or chin region of the subject; and a second retention band associated with the first cushion and configured to secure the first cushion to a top portion of a head H of the subject. In one embodiment, a second retention band consists of a front band section 400A configured to connect the first cushion 100 to the second cushion 200 and secures the device against the frontal portion of the head H; and a rear band section 400B configured to connect the first cushion 100 to the second cushion 200 and secures the device against the crown portion of the head H.

In other embodiments, the present disclosure provides a device for preventing a pressure injury associated with any ear of a subject. The device may comprise a first annular cushion comprising a first compressible ring that defines a first central void sized and shaped to receive a first ear of the subject; a second annular cushion comprising a second compressible ring that defines a second central void sized and shaped to receive a second ear of the subject; a chin strap configured to connect the first annular cushion and the second annular cushion; and a skull strap connection the first annular cushion and the second annular cushion. In the same embodiment, the chin strap and skull strap may be configured to collectively retain the first annular cushion and the second annular cushion against a head of the subject and about the first ear and the second ear, respectively.

In some embodiments, the present disclosure provides a method of preventing a pressure injury on an ear of a subject. The method comprises positioning the first cushion (e.g., the first annular cushion) of a device as disclosed herein about the first ear of the subject; positioning the first retention band under the chin of the subject; and positioning the second retention band around top portion of the head of the subject.

These and other embodiments are described in further detail herein below.

The figures depict various embodiments of this disclosure for purposes of illustration only. One skilled in the art will readily recognize from the following discussion that alternative embodiments of the structures and methods illustrated herein may be employed without departing from the principles of embodiments described herein.

### BRIEF DESCRIPTION OF THE DRAWINGS

FIG. 1 shows a perspective view of a device consistent with one embodiment of the present disclosure.

FIG. 2 shows a side perspective view of a device consistent with one embodiment of the present disclosure secured about the head of a subject.

FIG. 3 shows a front perspective view of a device consistent with one embodiment of the present disclosure secured about the head of a subject.

The figures depict various embodiments of this disclosure for purposes of illustration only. One skilled in the art will readily recognize from the following discussion that alternative embodiments of the structures and methods illustrated herein may be employed without departing from the principles of embodiments described herein.

### DETAILED DESCRIPTION

The present disclosure provides devices for preventing ear lobe pressure injuries in a subject, and methods of making and using same.

I. Devices for Preventing Pressure Injuries of the Ear

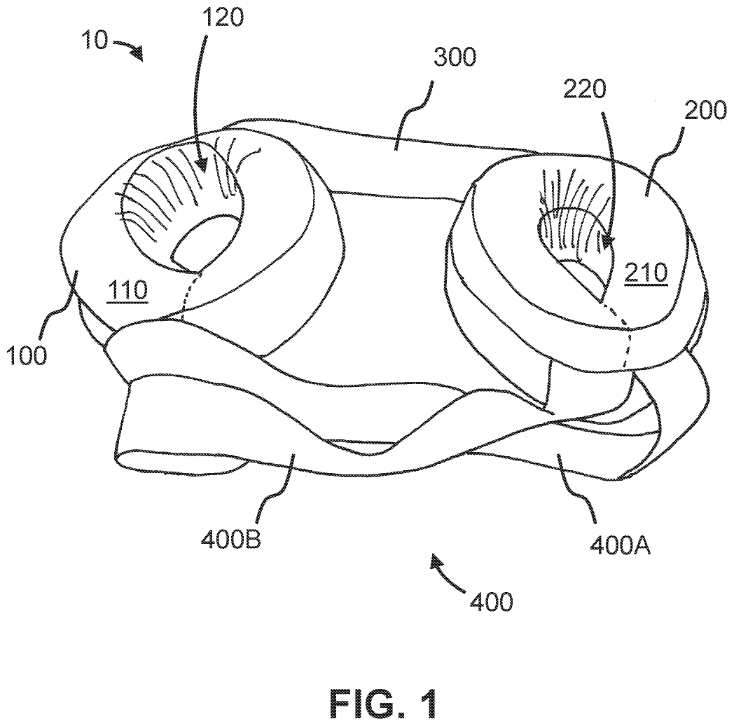

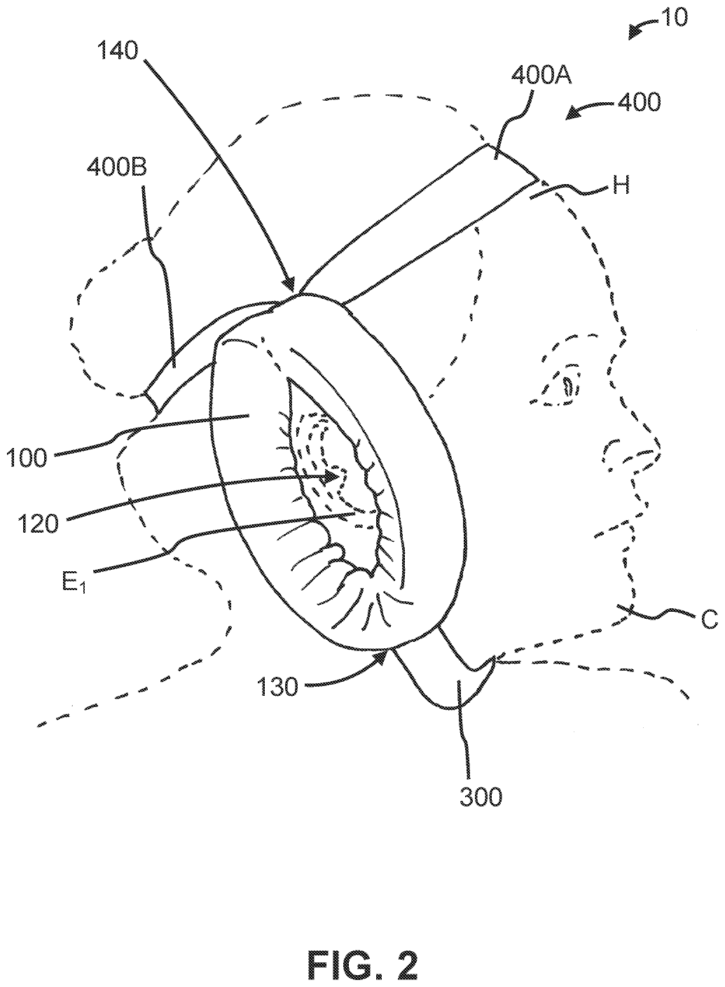

Referring generally to FIGS. 1-3, devices 10 consistent with the present disclosure include a first cushion 100, a first retention band 300 for securing the first cushion 100 to a subject's jaw or chin C, and a second retention band 400 for securing the first cushion 100 to a subject's head H or skull. In some embodiments, the device 10 further includes a second cushion 200.

The first cushion 100 is configured (e.g., sized and proportioned) to surround or substantially surround a first ear of the subject. The first cushion 100 may include a void 100, such as a central void, that is sized and shaped to accommodate the subject's ear (e.g., surrounds or substantially surrounds the subject's ear). In an embodiment, the first cushion 100 is constructed from any suitable compressible material, such as a foam, ball cottons, or other suitable fibrous material (e.g., a fire-retardant foam). The compressible material may be optionally surrounded by a cover 110 comprising, consisting essentially of, or consisting of fabric (e.g., a soft cotton cover). The first cushion 100 may include sufficient compressible material to prevent the subject's ear from bearing weight when the subject lies his or her head H down on the same side as that ear.

The compressible material may have an uncompressed thickness of about 2 inches to about 5 inches, for example about 2 inches, about 2.1 inches, about 2.2 inches, about 2.3 inches, about 2.4 inches, about 2.5 inches, about 2.6 inches, about 2.7 inches, about 2.8 inches, about 2.9 inches, about 3 inches, about 3.1 inches, about 3.2 inches, about 3.3 inches, about 3.4 inches, about 3.5 inches, about 3.6 inches, about 3.7 inches, about 3.8 inches, about 3.9 inches, about 4 inches, about 4.1 inches, about 4.2 inches, about 4.3 inches, about 4.4 inches, about 4.5 inches, about 4.6 inches, about 4.7 inches, about 4.8 inches, about 4.9 inches, or about 5 inches. In some embodiments, the compressible material may have a compressed thickness of about 1 inch to about 3 inches, for example about 1 inch, about 1.1 inches, about 1.2 inches, about 1.3 inches, about 1.4 inches, about 1.5 inches, about 1.6 inches, about 1.7 inches, about 1.8 inches, about 1.9 inches, about 2 inches, about 2.1 inches, about 2.2 inches, about 2.3 inches, about 2.4 inches, about 2.5 inches, about 2.6 inches, about 2.7 inches, about 2.8 inches, about 2.9 inches, or about 3 inches.

In some embodiments, the first retention band 300 (also referred to herein as the chin strap) is configured to secure the bottom ridge 130 of the first cushion 100 to the subject's jaw or chin C. In some embodiments, the first retention band 300 is elastic such that it draws the first cushion 100 towards the subject's head H. In another embodiment, the first retention band 300 is formed from a nonelastic material such that the device 10 fits loosely around a user's head H. In embodiments wherein the first cushion 100 includes a cover 110, the first retention band 300 may optionally be connected to the cover 110. In some embodiments, the first retention band 300 includes one or more fasteners (e.g., buttonholes 350) configured to enable adjustment of the operable length of the first retention band 300. In such an embodiment, the fastener may include any suitable configuration such as a buttonhole, Velcro, hook eye fasteners, zipper, or any other similar fastener system. In one embodiment, the first retention band 300 includes one or more buttonholes 350 configured to accept a button (not shown) disposed on the bottom ridge 130 of the first cushion 100. In another embodiment, the first cushion is secured to the first retention band 300 by a safety pin or other external fastener.

In some embodiments, the second retention band 400 (also referred to herein as the skull strap) is configured to secure the top ridge of the first cushion 100 to the top of the subject's head H (e.g., around the subject's crown). In some embodiments, the second retention band 400 is elastic such that it draws the first cushion 100 towards the subject's head H. In some such embodiments, the elastic second retention band 400 may comprise, consist essentially of, or consist of spandex, lycra, elastane, latex, rubber, isoprene, neoprene, nylon, cotton (e.g., jersey cotton), lace, or a similar stretchable material. In other embodiments, the second retention band 400 is formed from a nonelastic material such that the device fits loosely around a user's head H. In some such embodiments, the nonelastic material comprises, consists essentially of, or consists of plastic, leather, denim, polyester, or a similar unstretchable material. In embodiments wherein the first cushion 100 includes a cover 110, the second retention band 400 may optionally be connected to the cover 110. In some embodiments, the second retention band 400 includes a front band section 400A, and a rear band section 400B. In some embodiments, both the front band section 400A and rear band section 400B are formed of an elastic material that automatically adjusts to the size and shape of a user's head. In some such embodiments, the front band section 400A and/or the rear band section 400B may each independently comprise, consist essentially of, or consist of spandex, lycra, elastane, latex, rubber, isoprene, neoprene, nylon, cotton (e.g., jersey cotton), lace, or a similar stretchable material. In other embodiments, the front band section 400A and/or the rear band section 400B may each be independently formed from a nonelastic material such that the device 10 fits loosely around a user's head H. In some such embodiments, the nonelastic material comprises, consists essentially of, or consists of plastic, leather, denim, polyester, or a similar unstretchable material. In some embodiments, the second retention band 400 includes one or more fasteners (e.g., buttonholes 450) configured to enable adjustment of the operable length of the second retention band 400. In such an embodiment, the fastener may include any suitable configuration such as a buttonhole, Velcro, hook eye fasteners, zipper, or other similar fastener system. In one embodiment, the second retention band 400 includes one or more buttonholes 450 configured to accept a button (not shown) disposed on the top ridge 140 of the first cushion 100. In another embodiment, the first cushion 100 is secured to a second retention band 400 by a safety pin or other external fastener.

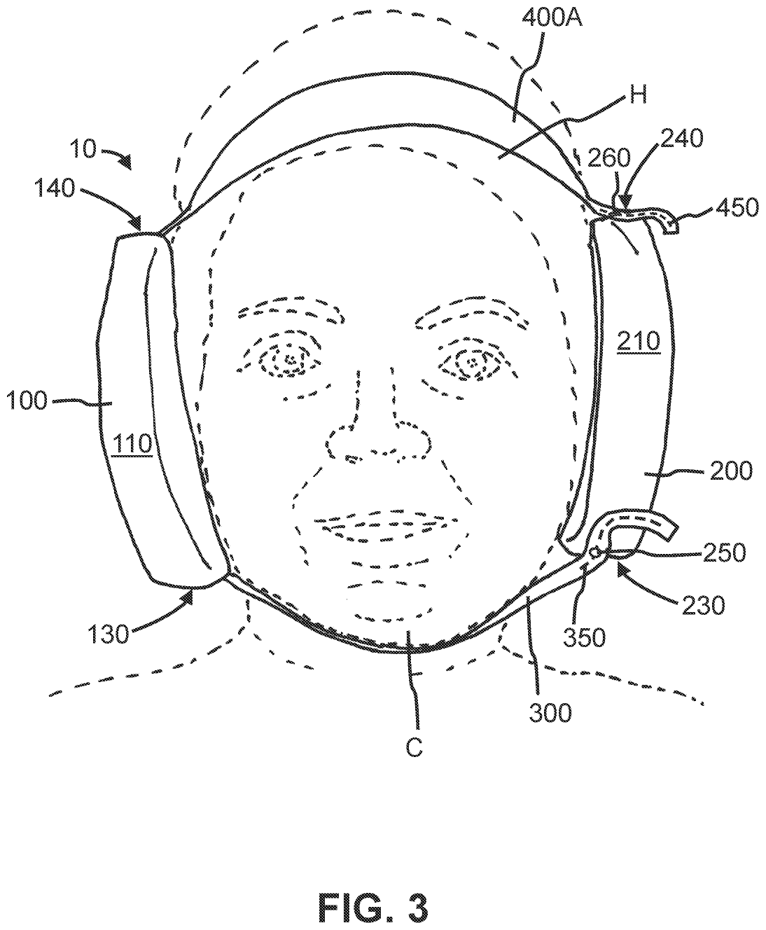

In some embodiments, the first retention band 300 includes one or more buttonholes 350 configured to mate with a button (not shown) disposed on the first cushion 100 (e.g., disposed near the top ridge 140 of the first cushion 100), similar to that shown in FIG. 3 with respect to the second cushion 200 of the two-cushion embodiments described in more detail below.

In some embodiments, the second retention band 400 (e.g., one or more of the front band section 400A and the rear band section 400B) includes one or more buttonholes 450 configured to mate with a button (not shown) disposed on the first cushion 100 (e.g., disposed near the top ridge 140 of the first cushion 100), similar to that shown in FIG. 3 with respect to the second cushion 200 of the two-cushion embodiments described in more detail below.

In some embodiments, the device 10 includes a second cushion 200. The second cushion 200 is configured (e.g., sized and proportioned) to surround or substantially surround an ear (not shown) of the subject. The second cushion 200 may include a void 200, such as a central void, that is sized and shaped to accommodate the subject's ear (e.g., surrounds or substantially surrounds the subject's ear). In an embodiment, the second cushion 200 is constructed from any suitable compressible material, such as a foam, ball cottons, or other suitable fibrous material (e.g., a fire-retardant foam). In some embodiments, the compressible material may be surrounded by a cover 210 comprising, consisting essentially of, or consisting of fabric (e.g., a soft cotton cover). The second cushion 200 may include sufficient compressible material to prevent the subject's ear from bearing weight when the subject lies his or her head H down on the same side as that ear.

The compressible material may have an uncompressed thickness of about 2 inches to about 5 inches, for example about 2 inches, about 2.1 inches, about 2.2 inches, about 2.3 inches, about 2.4 inches, about 2.5 inches, about 2.6 inches, about 2.7 inches, about 2.8 inches, about 2.9 inches, about 3 inches, about 3.1 inches, about 3.2 inches, about 3.3 inches, about 3.4 inches, about 3.5 inches, about 3.6 inches, about 3.7 inches, about 3.8 inches, about 3.9 inches, about 4 inches, about 4.1 inches, about 4.2 inches, about 4.3 inches, about 4.4 inches, about 4.5 inches, about 4.6 inches, about 4.7 inches, about 4.8 inches, about 4.9 inches, or about 5 inches. In some embodiments, the compressible material may have a compressed thickness of about 1 inch to about 3 inches, for example about 1 inch, about 1.1 inches, about 1.2 inches, about 1.3 inches, about 1.4 inches, about 1.5 inches, about 1.6 inches, about 1.7 inches, about 1.8 inches, about 1.9 inches, about 2 inches, about 2.1 inches, about 2.2 inches, about 2.3 inches, about 2.4 inches, about 2.5 inches, about 2.6 inches, about 2.7 inches, about 2.8 inches, about 2.9 inches, or about 3 inches.

In embodiments wherein the device 10 includes both a first cushion 100 and a second cushion 200, the first retention band 300 connects the bottom ridge 130 of the first cushion 100 to the bottom ridge 230 of the second cushion 200 via one or more fasteners (not shown) configured to enable adjustment of the operable length of the second retention band 300. In the same embodiments, the second retention band 400 connects the top ridge 140 of the first cushion 100 to the top ridge 240 of the second cushion 200 via one or more fasteners (not shown) configured to enable adjustment of the operable length of the second retention band 400. The fasteners may be any suitable fastener such as a buttonhole, Velcro, hook eye fasteners, zipper, or any other suitable fastener system. In one such embodiment, the first retention band 300 includes one or more buttonholes (not shown) configured to receive a button (not shown) disposed on the bottom ridge 230 of the second cushion to enable adjustment of the operable length of the first retention band 300. In the same embodiment, the first retention band 300 includes one or more buttonholes (not shown) configured to receive a button (not shown) disposed on the bottom ridge 130 of the first cushion 100 to enable adjustment of the operable length of the first retention band. In some embodiments, the second retention band 400 (e.g., the front band section 400A and/or the rear band section 400B) includes one or more buttonholes (not shown) configured to receive one or more buttons (not shown) disposed on the top ridge 240 of the second cushion 200. In some embodiments, the second retention band 400 (e.g., the front band section 400A and/or the rear band section 4008) includes one or more buttonholes (not shown) configured to receive one or more buttons (not shown) disposed on the top ridge 140 of the first cushion 100.

In another embodiment, the first retention band 300 includes one or more strips of hook-and-loop fastener (e.g., Velcro) configured to mate with a complementary hook-and-loop fastener (e.g., Velcro) strip disposed on the bottom ridge 230 of the second cushion to enable adjustment of the operable length of the first retention band 300. In the same embodiment, the second retention band 400 (e.g., the front band section 400A and/or the rear band section 400B) includes one or more strips of hook-and-loop fastener (e.g., Velcro) configured to mate with complementary hook-and-loop fastener (e.g., Velcro) disposed on the top ridge 240 of the second cushion 200. In the same embodiment, the first retention band 300 includes one or more strips of hook-and-loop fastener (e.g., Velcro) configured to mate with a complementary hook-and-loop fastener (e.g., Velcro strip) on the bottom ridge 130 of the first cushion 100 to enable adjustment of the operable length of the first retention band 300. In the same embodiment, the second retention band 400 (e.g., the front band section 400A and/or the rear band section 400B) includes one or more strips of hook-and-loop fastener (e.g., Velcro) configured to mate with complementary hook-and-loop fastener (e.g., Velcro) disposed on the top ridge 140 of the first cushion 100. In yet another embodiment the first retention band 300 is connected to the bottom ridge 230 of the second cushion 200 by a safety pin or other device. In the same embodiment a first retention band is attached to the bottom ridge 130 of the first cushion 100 by a safety pin or other device. In the same embodiment, the second retention band 400 (e.g., the front band section 400A and/or the rear band section 4008) is attached to the top ridge 240 of the second cushion 200 by a safety pin or other device. In the same embodiment, the second retention band 400 (e.g., the front band section 400A and/or the rear band section 400B) is attached to the top ridge 140 of the first cushion 100 by a safety pin or other device.

In some embodiments, the first retention band 300 includes one or more buttonholes 350 configured to mate with a button 250 disposed on the second cushion 200 (e.g., disposed near the bottom ridge 230 of the second cushion 200). In some embodiments, the first retention band 300 further includes one or more buttonholes 350 configured to mate with a button (not shown) disposed on the first cushion 100 (e.g., disposed near the top ridge 140 of the first cushion 100).

In some embodiments, the second retention band 400 (e.g., one or more of the front band section 400A and the rear band section 400B) includes one or more buttonholes 450 configured to mate with a button 260 disposed on the second cushion 200 (e.g., disposed near the top ridge 240 of the second cushion 200). In some embodiments, the second retention band 400 further includes one or more buttonholes 450 configured to mate with a button (not shown) disposed on the first cushion 100 (e.g., disposed near the top ridge 140 of the first cushion 100).

The retention bands described above enable a device 10 to be manufactured in bulk as the retention band(s) are adjustable to fit various sizes of users' heads. This is cost effective compared to, for example, making each device 10 from custom measurements for an individual user. For example, the device 10 can be made in a single size suitable for use with infants, children, small adults, and large adults, etc.

Alternatively, a device 10 consistent with the present disclosure may be sized and shaped specifically for use with a subject within a specific age or size range, such as an infant, a toddler, a child, a small adult, or a large adult. In such embodiments, the cushion 100/200 is sized for a specific size subject or for subjects within a specific range of sizes.

In some embodiments, the present disclosure provides a device 10 for preventing a pressure injury associated with an ear of a subject. In some embodiments, the device 10 comprises a first cushion 100 configured to substantially surround a first ear E₁ of the subject; a first retention band 300 associated with the first cushion 100 and configured to secure the first cushion 100 to a jaw or chin region C of the subject; and a second retention band 400 associated with the first cushion 100 and configured to secure the first cushion 100 to a top portion of a head H of the subject. In some embodiments, the device 10 further comprises a second cushion 200 configured to substantially surround a second ear (not shown) of the subject. In some embodiments, the first cushion 100 completely surrounds the first ear E₁. In some embodiments, the first cushion 100 comprises a void 120 sized and shaped to receive the first ear of the subject. In some embodiments, the second cushion 200 completely surrounds the second ear (not shown). In some embodiments, the second cushion 200 comprises a void 220 sized and shaped to receive the second ear of the subject (not shown). In some embodiments, the second retention band 400 comprises a front band section 400A configured to partially secure the first cushion 100 to a frontal portion of the head H, and a rear band section 400B configured to partially secure the first cushion 100 to a crown portion of the head H. In this embodiment the second band 400 (e.g., the front band section 400A and/or the rear band section 400B) is configured to be of adjustable length using any appropriate fastener so that the device may adjust to fit different head sizes. A further improvement would be to construct the second band 400 from an elastic material so that the device 10 is self-conforming to the wearer's head size without need for mechanical adjustment of the fasteners. Alternatively, the second band 400 may be composed of a nonelastic material so that the device 10 can be worn as tightly or loosely as the wearer chooses. In an embodiment the first retention band connects the bottom ridge 130 of a first cushion 100 to the bottom ridge 230 of a second cushion 200 by a fastener. In this embodiment the first band 300 is configured to be of adjustable length using any appropriate fastener so that the device may adjust to fit different head sizes. A further improvement would be to construct the first band 300 from an elastic material so that the device 10 is self-conforming to the wearer's head size without need for mechanical adjustment of the fasteners. Alternatively, the first band 300 may be composed of a nonelastic material so that the device 10 can be worn as tightly or as loosely as the wearer selects.

In some embodiments, the present disclosure provides a device 10 for preventing a pressure injury associated with any ear of a subject. The device 10 comprises a first annular cushion 100 comprising a first compressible ring that defines a first central void 120 sized and shaped to receive a first ear E₁ of the subject, and a second annular cushion 200 comprising a second compressible ring that defines a second central void 220 sized and shaped to receive a second ear (not shown) of the subject. The device 10 further includes a chin strap 300 configured to connect the first annular cushion 100 and the second annular cushion 200; and a skull strap 400 configured to connect the first annular cushion 100 and the second annular cushion 200. Further the device allows the chin strap C and the skull strap 400 to be configured collectively to retain the first annular cushion 100 and the second annular cushion 200 against a head H of the subject and about the first ear E 1 and the second ear (not shown), respectively.

In some embodiments, the first cushion 100 of a device 10 consistent with the present disclosure has a thickness of about 2 inches to about 5 inches. In some embodiments, the second cushion 200 has a thickness of about 2 inches to about 5 inches.

In some embodiments, the first cushion 100 of a device 10 consistent with the present disclosure comprises, consists essentially of, or consists of a compressible material, such as for example foam, surrounded by a cover 110. In some embodiments, the cover 110 comprises, consists essentially of, or consists of a fabric, for example a cotton, silk, nylon, linen, velvet or other suitable material selected by a wearer.

In some embodiments, the second cushion 200 of a device 10 consistent with the present disclosure comprises, consists essentially of, or consists of a compressible material, such as for example foam, surrounded by a cover 210. In some embodiments, the cover 220 comprises, consists essentially of, or consists of a fabric, for example a cotton, silk, nylon, linen, velvet or any other suitable material selected by a wearer.

II. Methods of Making Pressure Injury Prevention Devices

In some embodiments, the present disclosure provides a method of making a device 10 for preventing pressure injuries on an ear of a subject. In some embodiments, the method includes surrounding a length of a compressible material (e.g., foam) with a tubular cover 110/210, then joining the ends of the tubular cover to form an annular cushion 100/200. The first retention strap 300 is attached to the bottom ridge 130/230 of the annular cushion 100/200, and the second retention band 400 is attached to the top ridge 140/240 of the annular cushion 100/200. Optionally the second retention band 400 can be composed of two separate components. A front band section 400A connects the first cushion 100 to the second cushion 200 and secures the device against the frontal portion of the head H. A rear band section 400B connects the first cushion 100 to the second cushion 200 and secures the device against the crown portion of the head H. The bands are to be fixed at the appropriate fit; without causing any harm to the surrounding tissues using fasteners. These fasteners can be composed of any suitable mechanical device for securing a strap 300/400 to a cushion 100/200 at the desired length for example Velcro, hook and loop fasteners, button closures, etc.

In some embodiments, the disclosure provides a method of forming an annular cushion 100/200 wherein the annular cushion 100/200 is defined by having a central void 120 sized and shaped to receive a corresponding ear of the subject. The method further reduces the pressure placed on sensitive tissues increasing the devices effectiveness of preventing sores. In some embodiments, the user's ear is positioned substantially within or entirely within the central void 120.

In some embodiments, a method of making an annular cushion 100/200 consistent with the present disclosure includes forming a tubular cover 110/210 thereafter inserting compressible material in the lumen of the tubular cover 110/210, and thereafter closing the ends of the tubular cover 110/210. In this embodiment, the retention band(s) 300/400 may be secured to the cushion 100/200 before or after the compressible material is inserted into the tubular cover 110/210.

In other embodiments, a method of making an annular cushion 100/200 consistent with the present disclosure includes wrapping compressible material in cover material (e.g., cloth) and thereafter sealing (e.g., by sewing) the cover material around the compressible material. In this embodiment, the retention band(s) 300/400 may be secured to the cushion 100/200 at the same time the cover 110/210 is sealed about the compressible material.

III. Methods of Preventing Pressure Injuries

The present disclosure provides methods of using a device 10 as disclosed herein to prevent development of (e.g., prevent, delay development of, or attenuate the severity of) a pressure injury on an ear of a subject.

In a representative embodiment, the method comprises positioning the first cushion 100 of the device 10 about the first ear E₁ of the subject, and securing the first cushion 100 to the subject's head H using the first retention band 300 and the second retention band 400. In embodiments wherein the device 10 includes a second cushion 200, the method additionally comprises positioning the second cushion 200 about the second ear (not shown) of the subject.

In embodiments wherein the first retention band 300 includes one or more buttonholes 350, the method may additionally include mating a button (not shown) on the bottom ridge 130 of the first cushion 100, and/or mating a button 250 on the bottom ridge 230 of the second cushion 200, to a selected buttonhole 350 to appropriately secure the first cushion 100 (and optionally the second cushion 200) to the subject's head H. In other embodiments this method could be performed using an alternative fastener device like Velcro or hook and loop closures.

In embodiments wherein the second retention band 400 includes one or more buttonholes 450, the method may additionally include mating a button (not shown) on the top ridge 140 of the first cushion 100, and/or mating a button 260 on the top ridge 240 of the second cushion 200, to a selected buttonhole 450 to appropriately secure the first cushion 100 (and optionally the second cushion 200) to the subject's head H. In other embodiments this method could be performed using an alternative fastener device like Velcro or hook and loop closures.

In use, the skin of the ear(s) of the subject is protected against pressure injury development as the first cushion 100 enables the subject to lie his or her head down without the ear bearing substantial weight.

In some embodiments, the subject lacks capacity to position or reposition the device 10 on his or her own head H. In some such embodiments, the subject is comatose, has poor motor skills (e.g., poor fine motor skills), is unresponsive, is sedated, is on hospice care, or has a handicap or injury that prevents the subject from reaching the device 10 once positioned on the head H.

In some embodiments, the subject develops fewer and/or less severe pressure injuries on an ear after wearing the device for a period of time, compared to a similar subject that does not wear the device for the period of time. In some embodiments, the period of time is about one week, about two weeks, about three weeks, about one month, about two months, about three months, or more than three months.

### Examples

Example 1. A device for preventing a pressure injury associated with an ear of a subject, the device comprising:
a first cushion configured to substantially surround a first ear of the subject;
a first retention band associated with the first cushion and configured to secure the first cushion to a jaw or chin region of the subject; and
a second retention band associated with the first cushion and configured to secure the first cushion to a top portion of a head of the subject.

Example 2. The device of Example 1 further comprising a second cushion configured to substantially surround a second ear of the subject.

Example 3. The device of Example 1 or Example 2, wherein the first cushion completely surrounds the first ear.

Example 4. The device of any one preceding Example, wherein the first cushion comprises a void sized and shaped to receive the first ear of the subject.

Example 5. The device of any one of Examples 2-4, wherein the second cushion completely surrounds the second ear.

Example 6. The device of any one of Examples 2-5, wherein the second cushion comprises a void sized and shaped to receive the second ear of the subject.

Example 7. The device of any one preceding Example, wherein the second retention band comprises a front band section configured to partially secure the first cushion to a frontal portion of the head, and a rear band section configured to partially secure the first cushion to a crown portion of the head.

Example 8. A device for preventing a pressure injury associated with any ear of a subject, the device comprising:
a first annular cushion comprising a first compressible ring that defines a first central void sized and shaped to receive a first ear of the subject;
a second annular cushion comprising a second compressible ring that defines a second central void sized and shaped to receive a second ear of the subject;
a chin strap configured to connect the first annular cushion and the second annular cushion; and
a skull strap configured to connect the first annular cushion and the second annular cushion,
   wherein the chin strap and the skull strap are configured to collectively retain the first annular cushion and the second annular cushion against a head of the subject and about the first ear and the second ear, respectively.

Example 9. The device of any one preceding Example, wherein the first cushion has a thickness of about 2 inches to about 5 inches.

Example 10. The device of any one of Examples 2-9, wherein the second cushion has a thickness of about 2 inches to about 5 inches.

Example 11. The device of any one preceding Example, wherein the first cushion comprises, consists essentially of, or consists of a compressible foam surrounded by a cover.

Example 12. The device of Example 11, wherein the cover comprises, consists essentially of, or consists of a cotton fabric.

Example 13. The device of any one preceding Example, wherein the second cushion comprises, consists essentially of, or consists of a compressible foam surrounded by a cover.

Example 14. The device of Example 13, wherein the cover comprises, consists essentially of, or consists of a cotton fabric.

Example 15. A method of preventing a pressure injury on an ear of a subject, the method comprising:
positioning the first cushion of a device of any one of Examples 1-7 about the first ear of the subject;
positioning the first retention band under the chin of the subject; and
positioning the second retention band around top portion of the head of the subject.

Example 16. The method of Example 15, wherein the device is a device of any one of Examples 2-7, and wherein the method further comprises positioning the second cushion about the second ear of the subject.

Example 17. A method of preventing a pressure injury on an ear of a subject, the method comprising:
positioning the first annular cushion of a device of any one of Examples 8-14 about the first ear of the subject;
positioning the second annular cushion about the second ear of the subject;
positioning the chin strap under a chin of the subject; and
positioning the skull strap band around a head of the subject.

Example 18. The method of any one of Examples 15-17 further comprising determining that the subject is at risk for developing a pressure injury on an ear prior to the step of positioning a cushion about the first ear of the subject.

Example 19. The method of any one of Examples 15-18, wherein the subject develops fewer and/or less severe pressure injuries on an ear after wearing the device for a period of time, compared to a similar subject that does not wear the device for the period of time.

Example 20. The method of Example 19, wherein the period of time is about one week, about two weeks, about three weeks, about one month, about two months, about three months, or more than three months.

### CONCLUSION

The foregoing descriptions of specific embodiments of the present disclosure have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the device and its practical application, to thereby enable others skilled in the art to best utilize the device and various embodiments with various modifications as are suited to the particular use contemplated. It is intended that the scope of the present disclosure be defined by the Claims appended hereto and their equivalents.

It is to be understood that both the foregoing descriptions are exemplary and explanatory only, and are not restrictive of the methods and devices described herein. In this application, the use of the singular includes the plural unless specifically stated otherwise. Also, the use of “or” means “and/or” unless stated otherwise. Similarly, “comprise,” “comprises,” “comprising,” “include,” “includes” and “including” are not intended to be limiting.

All patents, patent applications, publications, and references cited herein are expressly incorporated by reference to the same extent as if each individual publication or patent application was specifically and individually indicated to be incorporated by reference.

What is claimed is:

1. A device for preventing a pressure injury associated with an ear of a subject, the device comprising:
a first annular cushion comprising a first compressible foam ring having a thickness of about 2 inches to about 5 inches and surrounded by a cover, wherein the first annular cushion defines a first central void sized and shaped to receive a first ear of the subject;
a second annular cushion comprising a second compressible foam ring having a thickness of about 2 inches to about 5 inches and surrounded by a cover, wherein the second annular cushion defines a second central void sized and shaped to receive a second ear of the subject;
a chin strap configured to connect the first annular cushion and the second annular cushion; and
a skull strap configured to connect the first annular cushion and the second annular cushion,
wherein the chin strap and the skull strap are configured to collectively retain the first annular cushion and the second annular cushion against a head of the subject and about the first ear and the second ear, respectively.

2. The device of claim 1, wherein the cover comprises, consists essentially of, or consists of a cotton fabric.

3. A device for preventing a pressure injury associated with an ear of a subject, the device comprising:
a first cushion configured to substantially surround a first ear of the subject;
a first retention band associated with the first cushion and configured to secure the first cushion to a jaw or chin region of the subject; and
a second retention band associated with the first cushion and configured to secure the first cushion to a top portion of a head of the subject.

4. The device of claim 3 further comprising a second cushion configured to substantially surround a second ear of the subject.

5. The device of claim 3, wherein the first cushion completely surrounds the first ear.

6. The device of claim 3, wherein the first cushion comprises a void sized and shaped to receive the first ear of the subject.

7. The device of claim 4, wherein the second cushion completely surrounds the second ear.

8. The device of claim 4, wherein the second cushion comprises a void sized and shaped to receive the second ear of the subject.

9. The device of claim 3, wherein the second retention band comprises a front band section configured to partially secure the first cushion to a frontal portion of the head, and a rear band section configured to partially secure the first cushion to a crown portion of the head.

10. A device for preventing a pressure injury associated with any ear of a subject, the device comprising:
a first annular cushion comprising a first compressible ring that defines a first central void sized and shaped to receive a first ear of the subject;
a second annular cushion comprising a second compressible ring that defines a second central void sized and shaped to receive a second ear of the subject;
a chin strap configured to connect the first annular cushion and the second annular cushion; and
a skull strap configured to connect the first annular cushion and the second annular cushion,
wherein the chin strap and the skull strap are configured to collectively retain the first annular cushion and the second annular cushion against a head of the subject and about the first ear and the second ear, respectively.

11. The device of claim 10, wherein the first cushion has a thickness of about 2 inches to about 5 inches.

12. The device of claim 10, wherein the second cushion has a thickness of about 2 inches to about 5 inches.

13. The device of claim 10, wherein the first cushion comprises, consists essentially of, or consists of a compressible foam surrounded by a cover.

14. The device of claim 13, wherein the cover comprises, consists essentially of, or consists of a cotton fabric.

15. The device of claim 10, wherein the second cushion comprises, consists essentially of, or consists of a compressible foam surrounded by a cover.

16. The device of claim 15, wherein the cover comprises, consists essentially of, or consists of a cotton fabric.

17. A method of preventing a pressure injury on an ear of a subject, the method comprising:
optionally determining that the subject is at risk for developing a pressure injury on an ear;
positioning the first annular cushion of a device of claim 1 about the first ear of the subject;
positioning the second annular cushion about the second ear of the subject;
positioning the chin strap under a chin of the subject; and
positioning the skull strap band around a head of the subject.

18. A method of preventing a pressure injury on an ear of a subject, the method comprising:
optionally determining that the subject is at risk for developing a pressure injury on an ear;
positioning the first cushion of a device of claim 3 about the first ear of the subject;
positioning the first retention band under the chin of the subject; and
positioning the second retention band around top portion of the head of the subject.

19. A method of preventing a pressure injury on an ear of a subject, the method comprising:
optionally determining that the subject is at risk for developing a pressure injury on an ear;
positioning the first annular cushion of a device of claim 10 about the first ear of the subject;
positioning the second annular cushion about the second ear of the subject;
positioning the chin strap under a chin of the subject; and
positioning the skull strap band around a head of the subject.
